(19) United States
(12) Patent Application Publication
(10) Pub. No.: US 2007/0160528 A1
(43) Pub. Date: Jul. 12, 2007

(54) Method to evaluate the likelehood of development of bone metastasis based on the determination of calcium binding proteins

(76) Inventor: Kenneth Wayne Culver, Mendham, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/548,525

(22) PCT Filed: Mar. 9, 2004
(86) PCT No.: PCT/EP04/02415

§ 371 Date: Feb. 6, 2007

Related U.S. Application Data
(60) Provisional application No. 60/453,331, filed on Mar. 10, 2003.
(60) Provisional application No. 60/464,822, filed on Apr. 23, 2003.

(51) Int. Cl. A61K 51/00; A61K 39/395; A61K 38/17

(52) U.S. Cl. 424/1.49; 424/155.1; 514/12

(57) ABSTRACT

This invention provides methods to determine which patients with cancer will develop bone metastasis by determining the presence or levels of calcium binding proteins in the blood or other tissues of these patients. These calcium binding proteins include MRP14. In addition, this invention provides methods for treating patients with cancer to reduce the development of bone metastasis. Also provided are kits by which to carry out these determinations.

### SUMMARY

This invention relates to the discovery of protein markers that predict whether a cancer is likely to metastasize to the patient's skeletal system (bone metastases).

### BACKGROUND

Metastatic cancers originate in one organ or part of the body and spread, often through the lymphatic or circulatory systems, to another part of the body that is not physically proximal to the site of origin. Metastasis of non-skeletal cancers into the patient's skeletal system often results in disabling bone cancer with poor prognosis.

A method to predict which non-skeletal cancers are likely to result in bone metastasis would enable medical professionals to intervene earlier in the course of the cancer with therapies that prevent or inhibit the spread of a cancer into the bone. This will result in the delay or prevention of disabling bone cancer and improved prognosis for the patient.

This invention provides a diagnostic method for predicting which cancers are likely to result in bone metastasis and to the use of the inventive diagnostic method to prevent or delay metastasis of a cancer to the skeleton.

### DETAILED DESCRIPTION

The present invention is based on experiments comparing peptides and proteins in plasma samples obtained from cancer patients who had metastasis to the skeleton with those found in plasma samples obtained from cancer patients with metastasis to the nodes and other sites, but not to the skeleton. These experiments demonstrate that a calcium binding protein, Migration Inhibitory Factor Related Protein 14 (MRP-14), is consistently expressed in cancer patients who have metastasis to bone, for example, about 68% of lung cancer patients who have bone metastasis, but not by those who did not have metastasis to bone. Further it is demonstrated that the level of MRP-14 in sera of patients with breast cancer with bone metastasis is increased when compared to the level of MRP-14 in sera from breast cancer patients without bone metastasis. From this, it is hypothesized that tumor cells that over-express calcium binding proteins shed and/or secrete them into the blood and that such tumor cells are better able to attach and grow into metastatic tumor deposits in bone compared to tumor that is not over-expressing calcium binding proteins.

This leads to the conclusion that one or more calcium binding proteins detected in the blood or cancerous tissue of a cancer patient is predictive of a patient who is at increased risk of developing bone metastasis.

Calcium binding proteins are known to those of skill in the art. Examples of calcium binding proteins include S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, Calcium- and Integrin-Binding Protein and MRP-14.

Thus, the present invention generally relates to a method for predicting whether a cancer is at increased risk of metastasis to the skeleton, which comprises determining whether the cancer cells over-express one or more calcium-binding proteins, especially wherein the calcium-binding protein is selected from MRP-14, S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, Calcium- and Integrin-Binding Protein.

The present invention further relates to inhibiting bone metastasis in cancers which over-express one or more calcium-binding proteins by treating the patient with a bone metastasis inhibiting treatment, such as zolendronic acid or a pharmaceutically acceptable salt thereof.

More particularly, this invention relates to a method of inhibiting bone metastasis in a patient diagnosed with a non-skeletal cancer, which comprises (a) testing a sample of tissue or body fluid from the patient for the presence of one or more calcium binding proteins, and, if one or more calcium binding proteins are detected, (b) treating the patient with a bone metastasis inhibiting therapy.

More preferably the invention relates to a method of inhibiting bone metastasis in a patient diagnosed with a non-skeletal cancer, which comprises (a) testing a cancerous tissue sample or blood from the patient for the presence of a calcium binding protein, and, if a calcium binding protein is detected, (b) treating the patient with a bone metastasis inhibiting treatment.

Members of the S100 protein family, such as MRP-14, S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, and Calcium- and Integrin-Binding Protein, are examples of calcium binding proteins that can be detected according to step (a).

The testing is generally conducted prior to clinical manifestation of metastasis, particularly bone metastasis, or detection of metastasis by conventional methods.

The non-skeletal cancer is in general any primary cancer not located in the patient's skeleton or bone. More specifically, the non-skeletal cancer is breast cancer, genitourinary cancer, lung cancer, gastrointestinal cancer, epidermoid cancer, melanoma, ovarian cancer, prostate cancer, pancreas cancer, neuroblastoma, head and/or neck cancer, bladder cancer, renal, brain or gastric cancer. In particular, the non-skeletal cancer may be breast, lung or prostate cancer.

Detection of the presence of a protein in plasma and tissue samples is carried out by methodology known in the art, for example by Western blot, ELISA and mass spectroscopy.

Preferably, the presence of one or more calcium binding proteins is detected by using one or more labeled probes specific for said one or more calcium binding proteins. Most preferably said labeled probe is an antibody or a radiolabeled binding partner. In an even more preferred embodiment said antibody is a monoclonal antibody.

In another embodiment of the invention the presence of one or more calcium binding proteins is determined by measuring the levels of expression of one or more genes encoding said one or more calcium binding proteins. Preferably said levels of expression are determined by measuring the level of mRNA using techniques selected from the group consisting of Microarray analysis, Northern blot analysis, reverse transcription PCR and real time quantitative PCR.

The tissue sample that is tested according to the present method is, for example, a sample obtained by surgery or biopsy or is a blood sample, particularly a plasma or serum sample. Preferably, the sample of tissue or body fluid that is tested is selected from the group consisting of a tissue biopsy, blood, serum, plasma, lymph, ascitic fluid, cystic fluid, urine, cerebro-spinal fluid (CSF), salvia or sweat.

In an especially useful embodiment of this invention the calcium-binding protein Migration Inhibitory Factor Related Protein 14 (MRP-14) is detected according to step (a). MRP-14 is known in the art under a number of synonyms such as: calgranulin B, P14, leukocyte L1 complex heavy chain, S100 calcium binding protein A9, calprotectin L1H subunit and myeloid related factor 14. It is a low molecular weight (MW=13,242 Daltons) calcium binding protein that contains two EF-hand motifs (helix-loop-helix). It can homodimerize or heterodimerize with a related protein, MRP-8. MRP-14 exists as a full length form and as a truncated form having an N-terminal truncation of 5 amino acids. In either case, the lead methionine is cleaved off and followed by acetylation. MRP-14 was first found in infiltrating macrophages during chronic or acute infiltration and is frequently upregulated in association with inflammatory disease.

Thus, an especially important embodiment of this invention relates to a method of inhibiting bone metastasis In a patient diagnosed with a non-skeletal cancer, which comprises (a) testing a cancerous tissue sample or plasma from the patient for the presence of Migration Inhibitory Factor Related Protein 14 (MRP-14), and, if MRP14 is detected, (b) treating the patient with a bone. metastasis inhibiting treatment. Further, the invention provides a method, in which the presence of MRP-14 is determined in a sample of tissue or body fluid, most preferably in plasma.

Detection of the presence MRP-14 in blood and tissue samples is carried out by methodology known in the art, for example by Western blot, ELISA and mass spectroscopy. Antibodies for detecting MRP-14 are commercially available, for example, MAC387, a mouse anti-human MRP-14 available from Bioprobe Indonesia. Such methodologies are useful for detecting MRP-14 according to the present invention. Methods for the detection of MRP-14 in tissues, such as plasma, have been described, for example, in Herndon et al, J. Lab. Clin. Med., 141(2):110-20 (2003) and Sinz A. et al, Electrophoresis, 23(19):3445-56 (2002).

Bone metastasis inhibiting therapies are know to those of skill in the art and include the range of antitumor therapies, including chemotherapy, treatment with bisphosphonates, treatment with biological agents, such as immunotherapy agents, and radiation therapy, alone or in combination.

In a preferred embodiment, the bone metastasis inhibiting therapy involves treatment with bone metastasis inhibiting pharmaceutical agents. Such agents are known to those of skill in the art and include bisphosphonate compounds, such as zolendronic acid, palmidronate, etidronate, tiludronate, alendonate, risedronate and the like. Zolendronic acid and palmidronate are especially useful, with zolendronic acid being preferred. Useful bone metastasis inhibiting agents include pharmaceutically acceptable salt and acid forms of the bisphosphonates. Methods for administering bisphosphonate agents are known in the art and will vary depending on the bisphosphonate.

Accordingly, the present invention further relates to a method of inhibiting bone metastasis in a patient diagnosed with a non-skeletal cancer, which comprises (a) testing a cancerous tissue sample or plasma from the patient for the presence of MRP-14, and, if migration inhibitory factor related protein 14 is detected, (b) treating the patient with an effective amount of a bisphosphonate, especially wherein the bisphosphonate is zolendronic acid, or a pharmaceutically acceptable salt thereof.

The present invention further relates to a method of predicting cancer patients at increased risk for bone metastasis, which comprises detecting a calcium binding protein, such as MRP-14, S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, Calcium- and Integrin- Binding Protein, especially MRP-14, in a sample of tissue or body fluid, preferably in a cancerous tissue sample or blood sample from the patient. Preferably, the presence of more than one calcium binding protein is detected in a sample of tissue or body fluid. In a preferred embodiment said sample of tissue or body fluid is selected from the group consisting of a tissue biopsy, blood, serum, plasma, lymph, ascitic fluid, cystic fluid, urine, cerebro-spinal fluid (CSF), salvia or sweat.

Accordingly, the invention provides a method for determining, which patient, diagnosed with a non-skeletal cancer, will be likely to develop bone metastasis; comprising the steps of a) obtaining a sample of tissue or body fluid from the said patient; b) determining whether the level of one or more calcium binding proteins is increased in the said sample of tissue or body fluid; c) determining that the said patient is in a high risk group for developing bone metastasis if the level of the one or more calcium binding proteins is increased; and d) determining that the said patient is in a low risk group for developing bone metastasis if the level of one or more calcium binding proteins is not increased.

A further embodiment of the invention relates to a method for determining, which patient, diagnosed with a non-skeletal cancer, will be likely to develop bone metastasis; comprising a) obtaining a sample of tissue or body fluid from the said patient; b) determining the presence of one or more calcium binding proteins in the said sample of tissue or body fluid; c) determining that the said patient is in a high risk group for developing bone metastasis if the presence of said one or more calcium binding proteins is detected; and d) determining that the said patient is in a low risk group for developing bone metastasis if said one or more calcium binding proteins are not detected.

Another aspect of the invention provides a method for determining, which patient, diagnosed with a non-skeletal cancer, will be likely to develop bone metastasis; comprising the steps a) determining in vitro or ex vivo whether the levels of one or more calcium binding proteins are increased in a sample of tissue or body fluid of said patient; b) determining that the said patient is in a high risk group for developing bone metastasis if the levels of the one or more calcium binding proteins are increased; and c) determining that the said patient is in a low risk group for developing bone metastasis if the levels of the one or more calcium binding proteins are not increased. A still further aspect of the invention relates to a method for determining, which patient, diagnosed with a non-skeletal cancer, will be likely to develop bone metastasis; comprising steps a) determining in vitro or ex vivo the presence of one or more calcium binding proteins in a sample of tissue or body fluid of said patient; b) determining that the said patient is in a high risk group for developing bone metastasis if the one or more calcium binding proteins are detected; and c) determining that the said patient is in a low risk group for developing bone metastasis if the one or more calcium binding protein are not detected.

The calcium binding protein is preferably selected from the group consisting of MRP-14, S100A to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, and Calcium- and Integrin-Binding Protein. The calcium binding protein is most preferably MRP-14.

Preferred embodiments provide that the levels of or the presence of one or more calcium binding proteins in the said sample of tissue or body fluid are determined by measuring the levels of the said one or more calcium binding protein by means of mass spectrometry. Alternatively, the levels of or the presence of the one or more calcium binding proteins may be determined by means of a reagent which specifically binds to the protein. In a preferred embodiment such reagent is a labeled probe specific for the said calcium binding protein. Most preferably, the reagent selected is an antibody such as a monoclonal antibody.

In some embodiments of the methods of this invention, the immunoassay described in U.S. Pat. Nos. 5,599,677 and 5,672,480, each of which is herein incorporated by reference, may be utilized. In other embodiments, proteins are detected by immunohistochemistry, as in Example 1 below.

Antibodies for Detection of Protein Levels

As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein or a fragment of the protein. For the production of antibodies to a protein or to a fragment of the protein, various host animals may be immunized by injection with the protein or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a calcium binding protein, or an antigenic functional derivative thereof. For the production of polyclonal antibodies, host animals, such as those described above, may be immunized by injection with the encoded protein, or a portion thereof, supplemented with adjuvants as also described above. Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature, Vol. 256, pp. 495-497 (1975); and U.S. Pat. No. 4,376,110), the human B-cell hybridoma technique (Kosbor et al., Immunology Today, Vol. 4, p. 72 (1983); Cole et al., Proc. Natl. Acad. Sci. USA, Vol. 80, pp. 2026-2030 (1983)), and the EBV-hybridoma technique (Cole et al., Monoclonal Antibodies and Cancer Therapy, Alan R. Liss, Inc., pp. 77-96 (1985)). Such antibodies may be of any immunoglobulin class, including IgG, IgM, IgE, IgA, IgD, and any subclass thereof.

The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule of appropriate antigen specificity, together with genes from a human antibody molecule of appropriate biological activity, can be used.

A chimeric antibody is a molecule in which different portions are derived from different animal species, such as those having a variable or hypervariable region derived from a murine mAb and a human immunoglobulin constant region. Alternatively, techniques described for the production of single-chain antibodies (U.S. Pat. No. 4,946,778; Bird, Science, Vol. 242, pp. 423-426 (1988); Huston et al., Proc. Nati. Acad. Sci. USA, Vol. 85, pp. 5879-5883 (1988); and Ward et al., Nature, Vol. 334, pp. 544-546 (1989)) can be adapted to produce differentially expressed gene-single chain antibodies. Single chain antibodies are formed by linking the heavy and light chain fragments of the Fv region via an amino acid bridge, resulting in a single-chain polypeptide. Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Altematively, Fab expression libraries may be constructed (Huse et al., Science, Vol. 246, pp. 1275-1281 (1989)) to allow rapid and easy identification of monoclonal Fab fragments with the desired specificity. The extent to which the known one or more calcium binding protein is present in a sample may then determined by immunoassay methods which utilize the antibodies described above. Such immunoassay methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody.

Any unreacted material is washed away, and the presence of the protein such as a calcium binding protein is determined by observation of a signal, or may be quantitated by comparing with a control sample containing known amounts of the protein. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody.

These techniques are well known to those skilled in the art, and the possibility of minor variations will be readily apparent. As used herein, “sandwich assay” is Intended to encompass all variations on the basic two-site technique. For the immunoassays of the present invention, the only limiting factor is that the labeled antibody be an antibody which is specific for the protein or a fragment thereof. The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate.

As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used.

It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of secreted protein or fragment thereof.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of Gene Expression

Another embodiment of the invention provides that the level of the one or more calcium binding proteins is determined by measuring the level of expression of one or more genes encoding said one or more calcium-binding proteins. The level of expression can be detected by standard methods as described further above. Preferably, the level of expression is determined by measuring the level of mRNA. Techniques for the detection of gene expression include, but are not limited to northern blots, RT-PCT, real time PCR, primer extension, RNase protection, RNA expression profiling and related techniques. These techniques are well known to those of skill in the art. Sambrook J et al., Molecular Cloning: A Laboratory Manual, Third Edition (Cold Spring Harbor Press, Cold Spring Harbor, 2000).

In particularly useful embodiments, the level of expression can be detected by techniques selected from the group consisting of Microarray analysis, Northern blot analysis, reverse transcription PCR and real time quantitative PCR.

Thus, in some embodiments, markers are detected at the level of cDNA or RNA.

As used herein, the term “gene expression biomarkers” shall mean any biologic marker which can indicate the rate or degree of gene expression of a specific gene including, but not limited to, mRNA, cDNA or the polypeptide expression product of the specific gene.

In some embodiments of the present invention, gene expression biomarkers are detected using a PCR-based assay. In yet other embodiments, reverse-transcriptase PCR (RT-PCR) is used to detect the expression of RNA. In RT-PCR, RNA is enzymatically converted to cDNA using a reverse-transcriptase enzyme. The cDNA is then used as a template for a PCR reaction. PCR products can be detected by any suitable method including, but not limited to, gel electrophoresis and staining with a DNA-specific stain or hybridization to a labeled probe.

In some embodiments, the quantitative RT-PCR with standardized mixtures of competitive templates method described in U.S. Pat. Nos. 5,639,606; 5,643,765; and 5,876,978, each of which is herein incorporated by reference, is utilized.

In preferred embodiments of the present invention, gene expression biomarkers are detected using a hybridization assay. In a hybridization assay, the presence or absence of a marker is determined based on the ability of the nucleic acid from the sample to hybridize to a complementary nucleic acid molecule, e.g., an oligonucleotide probe. A variety of hybridization assays are available.

In some embodiments, hybridization of a probe to the sequence of interest is detected directly by visualizing a bound probe, e.g., a Northern or Southern assay. See, e.g., Ausabel et al., eds., Current Protocols in Molecular Biology, John Wiley & Sons, NY (1991). In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide, is allowed to contact the membrane under low-, medium- or high-stringency conditions. Unbound probe is removed and the presence of binding is detected by visualizing the labeled probe.

In some embodiments, the DNA chip assay is a GeneChip (Affymetrix, Santa Clara, Calif.). See, e.g., U.S. Pat. Nos. 6,045,996; 5,925,525; and 5,858,659, each of which is herein incorporated by reference. The GeneChip technology uses miniaturized, high-density arrays of oligonucleotide probes affixed to a “chip”. Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementary, the Identity of the target nucleic acid applied to the probe array can be determined.

In other embodiments, a DNA microchip containing electronically captured probes (Nanogen, San Diego, Calif.) is utilized. See, e.g., U.S. Pat. Nos. 6,017,696; 6,068,818; and 6,051,380, each of which are herein incorporated by reference. Through the use of microelectronics, Nanogen's technology enables the active movement and concentration of charged molecules to and from designated test sites on its semiconductor microchip. DNA capture probes unique to a given gene expression biomarkers are electronically placed at, or “addressed” to, specific sites on the microchip. Since nucleic acid molecules have a strong negative charge, they can be electronically moved to an area of positive charge.

In still further embodiments, an array technology based upon the segregation of fluids on a flat surface (chip) by differences in surface tension (ProtoGene, Palo Alto, Calif.) is utilized. See, e.g., U.S. Pat. Nos. 6,001,311; 5,985,551; and 5,474,796, each of which is herein incorporated by reference. Protogene's technology is based on the fact that fluids can be segregated on a flat surface by differences In surface tension that have been imparted by chemical coatings. Once so segregated, oligonucleotide probes are synthesized directly on the chip by inkjet printing of reagents.

In yet other embodiments, a “bead array” is used for the detection of gene expression biomarkers (Illumina, San Diego, Calif.). See, e.g., PCT Publications WO 99/67641 and WO 00/39587, each of which is herein incorporated by reference. Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given marker. Batches of beads are combined to form a pool specific to the array. To perform an assay, the BEAD ARRAY is contacted with a prepared sample. Hybridization is detected using any suitable method.

In some preferred embodiments of the present invention, hybridization is detected by enzymatic cleavage of specific structures, e.g., INVADER™ assay, Third Wave Technologies. See, e.g., U.S. Pat. Nos. 5,846,717, 6,090,543; 6,001,567; 5,985,557; and 5,994,069, each of which is herein incorporated by reference. In some embodiments, hybridization of a bound probe is detected using a TaqMan assay (PE Biosystems, Foster City, Calif.). See, e.g., U.S. Pat. Nos. 5,962,233 and 5,538,848, each of which is herein incorporated by reference. The assay is performed during a PCR reaction. The TaqMan assay exploits the 5′-3′ exonuclease activity of DNA polymerases, such as AMPLITAQ DNA polymerase. A probe, specific for a given marker, is included in the PCR reaction. The probe consists of an oligonucleotide with a 5′-reporter dye, e.g., a fluorescent dye and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

Additional detection assays that are produced and utilized using the systems and methods of the present invention include, but are not limited to, enzyme mismatch cleavage methods, e.g., Variagenics (see U.S. Pat. Nos. 6,110,684; 5,958,692; and 5,851,770, herein incorporated by reference in their entireties); branched hybridization methods, e.g., Chiron (see U.S. Pat. Nos. 5,849,481; 5,710,264; 5,124,246; and 5,624,802, herein incorporated by reference in their entireties); rolling circle replication (see, e.g., U.S. Pat. Nos. 6,210,884 and 6,183,960, herein incorporated by reference in their entireties); NASBA (see, e.g., U.S. Pat. No. 5,409,818, herein incorporated by reference in its entirety); molecular beacon technology (see, e.g., U.S. Pat. No. 6,150,097, herein incorporated by reference in its entirety); E-sensor technology (see Motorola, U.S. Pat. Nos. 6,248,229; 6,221,583; 6,013,170; and 6,063,573, herein incorporated by reference in their entireties); cycling probe technology (see, e.g., U.S. Pat. Nos. 5,403,711; 5,011,769; and 5,660,988, herein incorporated by reference in their entireties); ligase chain reaction [see Barnay, Proc. Natl. Acad. Sci. USA, Vol. 88, pp. 189-93 (1991)]; and sandwich hybridization methods (see, e.g., U.S. Pat. No. 5,288,609, herein incorporated by reference in its entirety).

In some embodiments, mass spectroscopy is used to detect gene expression biomarkers. For example, in some embodiments, a MASSARRAY™ system (Sequenom, San Diego, Calif.) is used to detect gene expression biomarkers. See, e.g., U.S. Pat. Nos. 6,043,031; 5,777,324; and 5,605,798, each of which is herein incorporated by reference.

In some embodiments, the present invention provides kits for the identification, characterization and quantitation of gene expression biomarkers. In some embodiments, the kits contain antibodies specific for gene expression biomarkers, in addition to detection reagents and buffers. In other embodiments, the kits contain reagents specific for the detection of nucleic acid, e.g., oligonucleotide probes or primers. In preferred embodiments, the kits contain all of the components necessary to perform a detection assay, including all controls, directions for performing assays and any necessary software for analysis and presentation of results. In some embodiments, the kits contain instructions including a statement of intended use as required by the Environmental Protection Agency or U.S. Food and Drug Administration (FDA) for the labeling of in vitro diagnostic assays and/or of pharmaceutical or food products.

The experimental methods of this invention depend on measurements of cellular constituents. The cellular constituents measured can be from any aspect of the biological state of a cell. They can be from the transcriptional state, in which RNA abundances are measured, the translation state, in which protein abundances are measured, the activity state, in which protein activities are measured. The cellular characteristics can also be from mixed aspects, e.g., in which the activities of one or more proteins are measured along with the RNA abundances (gene expressions) of other cellular constituents. This section describes exemplary methods for measuring the cellular constituents in drug or pathway responses. This invention is adaptable to other methods of such measurement.

In some embodiments of this invention the transcriptional state of the other cellular constituents are measured. The transcriptional state can be measured by techniques of hybridization to arrays of nucleic acid or nucleic acid mimic probes, described in the next subsection, or by other gene expression technologies, described in the subsequent subsection. However measured, the result is data including values representing mRNA abundance and/or ratios, which usually reflect DNA expression ratios (in the absence of differences in RNA degradation rates).

In various alternative embodiments of the present invention, aspects of the biological state other than the transcriptional state, such as the translational state, the activity state or mixed aspects can be measured.

In all embodiments, measurements of the cellular constituents should be made in a manner that is relatively independent of when the measurement are made.

Transcriptional State Measurement

Preferably, measurement of the transcriptional state is made by hybridization to transcript arrays, which are described in this subsection. Certain other methods of transcriptional state measurement are described later in this subsection.

Transcript Arrays Generally

In some embodiments of the present invention use is made of “transcript arrays”, also called herein “microarrays”. Transcript arrays can be employed for analyzing the transcriptional state in a cell, and especially for measuring the transcriptional states of cancer cells.

In one embodiment, transcript arrays are produced by hybridizing detectably-labeled polynucleotides representing the mRNA transcripts present in a cell, e.g., fluorescently-labeled cDNA synthesized from total cell mRNA, to a microarray. A microarray is a surface with an ordered array of binding, e.g., hybridization, sites for products of many of the genes in the genome of a cell or organism, preferably most or almost all of the genes. Microarrays can be made in a number of ways, of which several are described below. However produced, microarrays share certain characteristics. The arrays are reproducible, allowing multiple copies of a given array to be produced and easily compared with each other. Preferably the microarrays are small, usually smaller than 5 cm² and they are made from materials that are stable under binding, e.g. nucleic acid hybridization, conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. In a specific embodiment, positionally-addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled, e.g., with a fluorophore, cDNA complementary to the total cellular mRNA is hybridized to a microarray, the site on the array corresponding to a gene, i.e., capable of specifically binding the product of the gene, that is not transcribed in the cell will have little or no signal, e.g., fluorescent signal, and a gene for which the encoded mRNA is prevalent will have a relatively strong signal.

Preparation of Microarrays

Microarrays are known in the art and consist of a surface to which probes that correspond in sequence to gene products, e.g., cDNAs, mRNAs, cRNAs, polypeptides and fragments thereof, can be specifically hybridized or bound at a known position. In one embodiment, the microarray is an array, i.e., a matrix, in which each position represents a discrete binding site for a product encoded by a gene, e.g., a protein or RNA, and in which binding sites are present for products of most or almost all of the genes in the organism's genome. In a preferred embodiment, the “binding site”, hereinafter “site”, is a nucleic acid or nucleic acid analogue to which a particular cognate cDNA can specifically hybridize. The nucleic acid or analogue of the binding site can be, e.g., a synthetic oligomer, a full-length cDNA, a less than full-length cDNA or a gene fragment.

Although in some embodiments the microarray contains binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to testing and confirming a biological network model of interest. A “gene” is identified as an open reading frame (ORF) of preferably at least 50, 75 or 99 amino acids from which a mRNA is transcribed in the organism, e.g., if a single cell, or in some cell in a multicellular organism. The number of genes in a genome can be estimated from the number of mRNAs expressed by the organism, or by extrapolation from a well-characterized portion of the genome. When the genome of the organism of Interest has been sequenced, the number of ORFs can be determined and mRNA coding regions identified by analysis of the DNA sequence. For example, the Saccharomyces cerevisiae genome has been completely sequenced and is reported to have approximately 6,275 ORFs longer than 99 amino acids. Analysis of these ORFs indicates that there are 5,885 ORFs that are likely to specify protein products. See Goffeau et al., Science, Vol. 274, pp. 546-567 (1996), which is incorporated by reference in its entirety for all purposes. In contrast, the human genome is estimated to contain approximately 10⁵ genes.

Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., PCR amplification of gene segments from genomic DNA, cDNA, e.g., by RT-PCR, or cloned sequences. PCR primers are chosen, based on the known sequence of the genes or cDNA, that result in amplification of unique fragments, i.e., fragments that do not share more than 10 bases of contiguous identical sequence with any other fragment on the microarray. Computer programs are useful in the design of primers with the required specificity and optimal amplification properties. See, e.g., Oligo pl version 5.0, National Biosciences. In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene fragment on the microarray will be between about 50 bp and about 2000 bp, more typically between about 100 bp and about 1000 bp, and usually between about 300 bp and about 800 bp in length. PCR methods are well-known and are described, e.g., in Innis et al., eds., PCR Protocols: A Guide to Methods and Applications, Academic Press Inc., San Diego, Calif. (1990), which is incorporated by reference in its entirety for all purposes. It will be apparent that computer-controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries. See Froehier et al., Nucleic Acid Res., Vol. 14, pp. 5399-5407 (1986); and McBride et al., Tetrahedron Lett., Vol. 24, pp. 245-248 (1983). Synthetic sequences are between about 15 bases and about 500 bases in length, more typically between about 20 bases and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid. See, e.g., Egholm et al., Nature, Vol. 365, pp. 566-568 (1993); and also U.S. Pat. No. 5,539,083.

In an alternative embodiment, the binding (hybridization) sites are made from plasmid or phage clones of genes, cDNAs, e.g., expressed sequence tags, or inserts therefrom. See Nguyen et al., Genomics, Vol. 29, pp. 207-209 (1995). In yet another embodiment, the polynucleotide of the binding sites is RNA.

Attaching Nucleic Acids to the Solid Surface

The nucleic acid or analogue are attached to a solid support, which may be made from glass, plastic, e.g., polypropylene and nylon, polyacrylamide, nitrocellulose or other materials. A preferred method for attaching the nucleic acids to a surface is by printing on glass plates, as is described generally by Schena et al., Science, Vol. 270, pp. 467-470 (1995). This method is especially useful for preparing microarrays of cDNA. See, also, DeRisi et al., Nat Genet., Vol. 14, pp. 457-460 (1996); Shalon et al., Genome Res., Vol. 6, pp. 639-645 (1996); and Schena et al., Proc. Natl. Acad. Sci. USA, Vol. 93, pp. 10539-11286 (1995). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ [see Fodor et al., Science, Vol. 251, pp. 767-773 (1991); Pease et al., Proc. Natl. Acad. Sci. USA, Vol. 91, No. 11, pp. 5022-5026 (1994); Lockhart et al., Nat. Biotechnol., Vol. 14, p. 1675 (1996); and U.S. Pat. Nos. 5,578,832; 5,556,752; and 5,510,270, each of which is incorporated by reference in its entirety for all purposes] or other methods for rapid synthesis and deposition of defined oligonucleotides [see Blanchard et al., Biosens. Bioelectron., Vol.11, pp. 687-690 (1996)]. When these methods are used, oligonucleotides, e.g., 20 mers, of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking, may also be used. See Maskos and Southern, Nucleic Acids Res., Vol. 20, pp. 1679-1684 (1992). In principal, any type of array, e.g., dot blots on a nylon hybridization membrane [see Sambrook et al., Molecular Cloning—A Laboratory Manual, 2^{nd} Edition, Vols. 1-3, Cold Spring Harbor Laboratory, Cold Spring Harbor, N.Y. (1989), which is incorporated in its entirety for all purposes], could be used, although, as will be recognized by those of skill in the art, very small arrays will be preferred because hybridization volumes will be smaller.

Generating Labeled Probes

Methods for preparing total and poly(A)⁺ RNA are well-known and are described generally in Sambrook et al. (1989), supra. In one embodiment, RNA is extracted from cells of the various types of interest in this invention using guanidinium thiocyanate lysis followed by CsCl centrifugation. See Chirgwin et al., Biochemistry, Vol. 18, pp. 5294-5299 (1979). Poly(A)⁺ RNA is selected by selection with oligo-dT cellulose. See Sambrook et al. (1989), supra. Cells of interest include wild-type cells, drug-exposed wild-type cells, cells with modified/perturbed cellular constituent(s), and drug-exposed cells with modified/perturbed cellular constituent(s).

Labeled cDNA is prepared from mRNA by oligo dT-primed or random-primed reverse transcription, both of which are well-known in the art. See, e.g., Klug and Berger, Methods Enzymol., Vol. 152, pp. 316-325 (1987). Reverse transcription may be carried out in the presence of a dNTP conjugated to a detectable label, most preferably a fluorescently-labeled dNTP. Alternatively, isolated mRNA can be converted to labeled antisense RNA synthesized by in vitro transcription of double-stranded cDNA in the presence of labeled dNTPs. See Lockhart et al. (1996), supra, which is incorporated by reference in its entirety for all purposes. In altemative embodiments, the cDNA or RNA probe can be synthesized in the absence of detectable label and may be labeled subsequently, e.g., by incorporating biotinylated dNTPs or rNTP, or some similar means, e.g., photo-cross-linking a psoralen derivative of biotin to RNAs, followed by addition of labeled streptavidin, e.g., phycoerythrin-conjugated streptavidin or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others. See, e.g., Kricka, Nonisotopic DNA Probe Techniques, Academic Press, San Diego, Calif. (1992). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used. See Zhao et al., Gene, Vol. 156, p. 207 (1995); and Pietu et al., Genome Res., Vol. 6, p. 492 (1996). However, because of scattering of radioactive particles, and the consequent requirement for widely-spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides, e.g., 0.1 mM Rhodamine 110 UTP (Perken Elmer Cetus) or 0.1 mM Cy3 dUTP (Amersham), with reverse transcriptase, e.g., SuperScript™. II, LTI Inc., at 42° C. for 60 minutes.

Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is ≦25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls. See, e.g., Shalon et al. (1996), supra; and Chee et al., supra.

Optimal hybridization conditions will depend on the length, e.g., oligomer vs. polynucleotide >200 bases; and type, e.g., RNA, DNA and PNA, of labeled probe and immobilized polynucleotide or oligonucleotide. General parameters for specific, i.e., stringent, hybridization conditions for nucleic acids are described in Sambrook et al. (1996), supra; and Ausubel et al., Current Protocols in Molecular Biology, Greene Publishing and Wiley-Interscience, NY (1987), which is incorporated in its entirety for all purposes. When the cDNA microarrays of Schena et al. are used, typical hybridization conditions are hybridization in 5×SSC plus 0.2% SDS at 65° C. for 4 hours followed by washes at 25° C. in low-stringency wash buffer (1×SSC plus 0.2% SDS) followed by 10 minutes at 25° C. in high-stringency wash buffer (0.1×SSC plus 0.2% SDS). See Shena et al., Proc. Natl. Acad. Sci. USA, Vol. 93, p. 10614 (1996). Useful hybridization conditions are also provided. See, e.g., Tijessen, Hybridization With Nucleic Acid Probes, Elsevier Science Publishers B.V. (1993); and Kricka (1992), supra.

Signal Detection and Data Analysis

When fluorescently-labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously. See Shalon et al. (1996), supra, which is incorporated by reference in its entirety for all purposes. In a preferred embodiment, the arrays are scanned with a laser fluorescent scanner with a computer-controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al. (1996), supra and in other references cited herein. Alternatively, the fiber-optic bundle described by Ferguson et al., Nat Biotechnol., Vol. 14, pp. 1681-1684 (1996), may be used to monitor mRNA abundance levels at a large number of sites simultaneously.

Signals are recorded and, in a preferred embodiment, analyzed by computer, e.g., using a 12-bit analog to digital board. In one embodiment the scanned image is de-speckled using a graphics program, e.g., Hijaak Graphics Suite, and then analyzed using an image gridding program that creates a spreadsheet of the average hybridization at each wavelength at each site. If necessary, an experimentally determined correction for “cross talk” (or overlap) between the channels for the two fluorophores may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion or any other tested event.

Preferably, In addition to identifying a perturbation as positive or negative, it is advantageous to determine the magnitude of the perturbation. This can be carried out by methods that will be readily apparent to those of skill in the art.

Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers [see, e.g., EP 0 534858 A1 (1992), Zabeau et al.], or methods selecting restriction fragments with sites closest to a defined mRNA end [see, e.g., Prashar et al., Proc. Natl. Acad. Sci. USA, Vol. 93, pp. 659-663 (1996)]. Other methods statistically sample cDNA pools, such as by sequencing sufficient bases, e.g., 20-50 bases, in each of multiple cDNAs to identify each cDNA, or by sequencing short tags, e.g., 9-10 bases, which are generated at known positions relative to a defined mRNA end pathway pattem. See, e.g., Velculescu, Science, Vol. 270, pp. 484-487 (1995).

Measurement of Other Aspects

In various embodiments of the present invention, aspects of the biological state other than the transcriptional state, such as the translational state, the activity state or mixed aspects can be measured in order to obtain drug and pathway responses. Details of these embodiments are described in this section.

Translational State Measurements

Measurement of the translational state may be performed according to several methods. For example, whole genome monitoring of protein, i.e., the “proteome” [see Goffeau et al. (1996), supra], can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well-known. See, e.g., Harlow and Lane, Antibodies: A Laboratory Manual, Cold Spring Harbor, N.Y. (1988), which is incorporated in its entirety for all purposes. In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., Gel Electrophoresis of Proteins: A Practical Approach, IRL Press, NY (1990); Shevchenko et al., Proc. Natl. Acad. Sci. USA, Vol. 93, pp. 1440-1445 (1996); Sagliocco et al., Yeast, Vol. 12, pp. 1519-1533 (1996); Lander, Science, Vol. 274, pp. 536-539 (1996). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells, e.g., in yeast; exposed to a drug or in cells modified by, e.g., deletion or over-expression of a specific gene.

Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical or physical means appropriate to the particular activity being characterized. Where the activity involves a chemical transformation, the cellular protein can be contacted with the natural substrates and the rate of transformation measured. Where the activity involves association in multimeric units, e.g., association of an activated DNA-binding complex with DNA, the amount of associated protein or secondary consequences of the association, such as amounts of mRNA transcribed, can be measured. Also, where only a functional activity is known, e.g., as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

Calcium Binding Protein Levels

As used herein, the level of one or more calcium binding proteins is “increased” when the level of the one or more proteins or the level of mRNA encoding said one or more proteins shows at least a 1.5-fold difference (ie., higher) in the level of protein or mRNA as compared to a patient, diagnosed with a non-skeletal cancer and not being affected by bone metastasis. Preferably said difference is a difference of at least; 1.5 fold, 2-fold, 3-fold, 5-fold, 10-fold, 30-fold, 70-fold or 100-fold when compared to a patient not suffering from bone metastasis.

Most preferably, a patient is determined as being in a low risk group for developing bone metastasis if no calcium binding protein detected. According to one preferred embodiment of the invention, a patient diagnosed with a non-skeletal cancer, is classified as being in a low risk group for developing bone metastasis if no MRP-14 is detected in a sample of tissue or body fluid such as in a plasma sample.

Another aspect of the invention relates to a method for screening for an agent useful in treating bone metastasis in a patient diagnosed with a non-skeletal cancer, comprising (a) administering a candidate agent to a non-human test animal which is predisposed to be affected or being affected by bone metastasis; (b) administering the candidate agent of (a) to a matched control non-human animal not predisposed to be affected or being affected by bone metastasis; (c) determining the levels of one or more calcium-binding proteins in a sample obtained from the animal of (a) and (b); and (d) comparing the levels determined in (c), wherein a decrease in the levels of the one or more calcium-binding proteins indicates that the candidate agent is an agent useful in treating bone metastasis.

In a preferred embodiment, the one or more calcium-binding protein is selected from MRP-14, S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, Calcium- and Integrin-Binding Protein. Most preferably the calcium binding protein is MRP-14.

According to a preferred embodiment said sample is a sample of tissue or body fluid, most preferably, it is a plasma sample. The level of the one or more calcium binding proteins may be determined by mass spectroscopy, by Western blot or ELISA using a labeled probe specific for the one or more calcium binding protein, such as an labeled antibody, preferably a monoclonal antibody or a radiolabeled binding partner. Alternatively, the level of the one or more calcium binding proteins may be determined by measuring the level of expression of one or more genes encoding said one or more calcium-binding proteins, for example by the use of Microarray analysis, Northern blot analysis, reverse transcription PCR and real time quantitative PCR.

As used herein, the term “decrease” refers to a statistically significant difference in measured levels or a difference of at least 1.5-fold in the level of the one or more calcium-binding proteins measured for a sample from the test animal when compared to the level determined for a sample of the non-human test animal. Preferably said alteration is a difference of at least 2-fold, 3-fold, 5-fold, 10-fold, 30-fold or 100-fold.

Agent Useful in Treating Bone Metastasis

In other embodiments of the invention, the agent useful in treating bone metastasis is selected from the group consisting of antisense nucleotides, ribozymes and double stranded RNAs, small molecules, antibodies or other means of modifying the abundance or activity of RNA, DNA or proteins.

Methods of Modifying RNA Abundances or Activities

Methods of modifying RNA abundances and activities currently fall within three classes: ribozymes, antisense species and RNA aptamers. See Good et al., Gene Ther., Vol. 4, No. 1, pp. 45-54 (1997). Controllable application or exposure of a cell to these entities permits controllable perturbation of RNA abundances.

Ribozymes

Ribozymes are RNAs which are capable of catalyzing RNA cleavage reactions. See Cech, Science, Vol. 236, pp. 1532-1539 (1987); PCT International Publication WO 90/11364 (1990); Sarver et al., Science, Vol. 247, pp. 1222-1225 (1990). “Hairpin” and “hammerhead” RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA. See Haseloff et al., Nature, Vol. 334, pp. 585-591 (1988); Koizumi et al., FEBS Lett., Vol. 228, pp. 228-230 (1988); and Koizumi et al., FEBS Lett., Vol. 239, pp. 285-288 (1988). Ribozyme methods involve exposing a cell to, inducing expression in a cell, etc. of such small RNA ribozyme molecules. See Grassi and Marini, Annals of Med., Vol. 28, No. 6, pp. 499-510 (1996); and Gibson, Cancer Meta. Rev., Vol. 15, pp. 287-299 (1996).

Ribozymes can be routinely expressed in vivo in sufficient number to be catalytically effective in cleaving mRNA, and thereby modifying mRNA abundances in a cell. See Cotton et al., EMBO J., Vol. 8, pp. 3861-3866 (1989). In particular, a ribozyme coding DNA sequence, designed according to the previous rules and synthesized, e.g., by standard phosphoramidite chemistry, can be ligated into a restriction enzyme site in the anticodon stem and loop of a gene encoding a tRNA, which can then be transformed into and expressed in a cell of interest by methods routine in the art.

Preferably, an inducible promoter, e.g., a glucocorticoid or a tetracycline esponse element, is also introduced into this construct so that ribozyme expression can be selectively controlled. For saturating use, a highly and constituently active promoter can be used. tDNA genes, i.e., genes encoding tRNAs, are useful in this application because of their small size, high rate of transcription and ubiquitous expression in different kinds of tissues. Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly, the abundance of virtually any RNA species in a cell can be modified or perturbed.

Antisense Molecules

In another embodiment, activity of a target RNA (preferably mRNA) species, specifically its rate of translation, can be controllably inhibited by the controllable application of antisense nucleic acids. Application at high levels results in a saturating inhibition. An “antisense” nucleic acid as used herein refers to a nucleic acid capable of hybridizing to a sequence-specific, e.g., non-poly A, portion of the target RNA, e.g., its translation initiation region, by virtue of some sequence complementary to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides, ranging from 6 oligonucleotides to about 200 oligonucleotides. In specific aspects, the oligonucleotide is at least 10 nucleotides, at least 15 nucleotides, at least 100 nucleotides or at least 200 nucleotides. The oligonucleotides can be DNA or RNA or chimeric mixtures or derivatives or modified versions thereof, single-stranded or double-stranded. The oligonucleotide can be modified at the base moiety, sugar moiety or phosphate backbone. The oligonucleotide may include other appending groups, such as peptides, or agents facilitating transport across the cell membrane [see, e.g., Letsinger et al., Proc. Natl. Acad. Sci. USA, Vol. 86, pp. 6553-6556 (1989); Lemaitre et al., Proc. Natl. Acad. Sci. USA, Vol. 84, pp. 648-652 (1987); and PCT Publicaton No. WO 88/09810 (1988)], hybridization-triggered cleavage agents [see, e.g., Krol et al., BioTechniques, Vol. 6, pp. 958-976 (1988)] or intercalating agents [see, e.g., Zon, Pharm. Res., Vol. 5, No. 9, pp. 539-549 (1988)].

In a preferred aspect of the invention, an antisense oligonucleotide is provided, preferably as single-stranded DNA. The oligonucleotide may be modified at any position on its structure with constituents generally known in the art.

The antisense oligonucleotides may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w and 2,6-diaminopurine.

In another embodiment, the oligonucleotide comprises at least one modified sugar moiety selected from the group including, but not limited to, arabinose, 2-fluoroarabinose, xylulose and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester and a formacetal or analog thereof.

In yet another embodiment, the oligonucleotide is a 2-a-anomeric oligonucleotide. An a-anomeric oligonucleotide forms specific double-stranded hybrids with complementary RNA in which, contrary to the usual B-units, the strands run parallel to each other. See Gautier et al., Nucl. Acids Res., Vol. 15, pp. 6625-6641 (1987).

The oligonucleotide may be conjugated to another molecule, e.g., a peptide, hybridization triggered cross-linking agent, transport agent, hybridization-triggered cleavage agent, etc.

The antisense nucleic acids of the invention comprise a sequence complementary to at least a portion of a target RNA species. However, absolute complementary, although preferred, is not required. A sequence “complementary to at least a portion of an RNA”, as referred to herein, means a sequence having sufficient complementary to be able to hybridize with the RNA, forming a stable duplex; in the case of double-stranded antisense nucleic acids, a single strand of the duplex DNA may thus be tested or triplex formation may be assayed. The ability to hybridize will depend on both the degree of complementary and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

Oligonucleotides of the invention may be synthesized by standard methods known in the art, e.g., by use of an automated DNA synthesizer, such as are commercially-available from Biosearch, Applied Biosystems, etc. As examples, phosphorothioate oligonucleotides may be synthesized by the method of Stein et al., Nucl. Acids Res., Vol. 16, p. 3209 (1988), methylphosphonate oligonucleotides can be prepared by use of controlled pore glass polymer supports, etc. See Sarin et al., Proc. Natl. Acad. Sci. USA, Vol. 85, pp. 7448-7451 (1988). In another embodiment, the oligonucleotide is a 2′-0-methylribonucleoude [see Inoue et al., Nucl. Acids Res., Vol. 15, pp. 6131-6148 (1987)] or a chimeric RNA-DNA analog [see Inoue et al., FEBS Lett., Vol. 215, pp. 327-330 (1987)].

The synthesized antsense oligonucleotides can then be administered to a cell in a controlled or saturating manner. For example, the antisense oligonucleotides can be placed in the growth environment of the cell at controlled levels where they may be taken up by the cell. The uptake of the antisense oligonucleotides can be assisted by use of methods well-known in the art.

Antisense Molecules Expressed Intracellularly

In an alternative embodiment, the antisense nucleic acids of the invention are controllably expressed intracellularly by transcription from an exogenous sequence. If the expression is controlled to be at a high level, a saturating perturbation or modification results. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral or others known in the art, used for replication and expression in mammalian cells.

Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to, the SV40 early promoter region [see Bemoist and Chambon, Nature, Vol. 290, pp. 304-310 (1981)], the promoter contained in the 3′ long terminal repeat of Rous sarcoma virus [see Yamamoto et al., Cell, Vol. 22, pp. 787-797 (1980)], the herpes thymidine kinase promoter [see Wagner et al., Proc. Natl. Acad. Sci. USA, Vol. 78, pp. 1441-1445 (1981)], the regulatory sequences of the metallothionein gene, etc. [see Brinster et al., Nature, Vol. 296, pp. 3942 (1982)].

Therefore, antisense nucleic acids can be routinely designed to target virtually any mRNA sequence, and a cell can be routinely transformed with or exposed to nucleic acids coding for such antisense sequences such that an effective and controllable or saturating amount of the antisense nucleic acid is expressed. Accordingly the translation of virtually any RNA species in a cell can be modified or perturbed.

RNA Aptamers

Finally, in a further embodiment, RNA aptamers can be introduced into or expressed in a cell. RNA aptamers are specific RNA ligands for proteins, such as for Tat and Rev RNA [see Good et al. (1997), supra] that can specifically Inhibit their translation.

Methods of Modifying Protein Abundances

Methods of modifying protein abundances include, inter alla, those altering protein degradation rates and those using antibodies, which bind to proteins affecting abundances of activities of native target protein species. Increasing (or decreasing) the degradation rates of a protein species decreases (or increases) the abundance of that species. Methods for increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature, e.g., 37° C., and which is hidden to prevent rapid degradation at a lower temperature, e.g., 23° C. See Dohmen et al., Science, Vol. 263, pp. 1273-1276 (1994). Such an exemplary degron is Arg-DHFR^{ts}, a variant of murine dihydrofolate reductase in which the N-terminal Val is replaced by Arg and the Pro at position 66 is replaced with Leu.

According to this method, e.g., a gene for a target protein, P, is replaced by standard gene targeting methods known in the art [see Lodish et al., Molecular Biology of the Cell, W. H. Freeman and Co., NY, especially Chapter 8 (1995)] with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFR^{ts} are not exposed, ubiquitination of the fusion protein does not occur, degradation is slow and active target protein levels are high. At higher temperatures (in the absence of methotrexate), lysines internal to Arg-DHFR^{ts} are exposed, ubiquitination of the fusion protein occurs, degradation is rapid and active target protein levels are low. This technique also permits controllable modification of degradation rates since heat activation of degradation is controllably blocked by exposure methotrexate. This method is adaptable to other N-terminal degrons which are responsive to other inducing factors, such as drugs and temperature changes.

Modifying Protein Activity with Antibodies

Target protein activities can also be decreased by (neutralizing) antibodies. By providing for controlled or saturating exposure to such antibodies, protein abundances/activities can be modified or perturbed in a controlled or saturating manner. For example, antibodies to suitable epitopes on protein surfaces may decrease the abundance, and thereby indirectly decrease the activity, of the wild-type active form of a target protein by aggregating active forms into complexes with less or minimal activity as compared to the wild-type unaggregated wild-type form.

Alternately, antibodies may directly decrease protein activity by, e.g., interacting directly with active sites or by blocking access of substrates to active sites. Conversely, in certain cases, (activating) antibodies may also interact with proteins and their active sites to increase resulting activity. In either case, antibodies (of the various types to be described) can be raised against specific protein species (by the methods to be described) and their effects screened. The effects of the antibodies can be assayed and suitable antibodies selected that raise or lower the target protein species concentration and/or activity. Such assays involve introducing antibodies into a cell (see below) and assaying the concentration of the wild-type amount or activities of the target protein by standard means, such as immunoassays, known in the art. The net activity of the wild-type form can be assayed by assay means appropriate to the known activity of the target protein.

Antibodies can be introduced into cells in numerous fashions, including, e.g., microinjection of antibodies into a cell [see Morgan et al., Immunol. Today, Vol. 9, pp. 84-86 (1988)] or transforming hybridoma mRNA encoding a desired antibody into a cell [see Burke et al., Cell, Vol. 36, pp. 847-858 (1984)]. In a further technique, recombinant antibodies can be engineering and ectopically expressed in a wide variety of non-lymphoid cell types to bind to target proteins, as well as to block target protein activities. See Biocca et al., Trends Cell Biol., Vol. 5, pp. 248-252 (1995). Expression of the antibody is preferably under control of a controllable promoter, such as the Tet promoter, or a constitutively active promoter (for production of saturating perturbations). A first step is the selection of a particular monoclonal antibody with appropriate specificity to the target protein (see below). Then sequences encoding the variable regions of the selected antibody can be cloned into various engineered antibody formats, including, e.g., whole antibody, Fab fragments, Fv fragments, single-chain Fv (ScFv) fragments (V_{H} and V_{L} regions united by a peptide linker), diabodies (two associated ScFv fragments with different specificities) and so forth. See Hayden et al., Curr. Opin. Immunol., Vol. 9, pp. 210-212 (1997).

Intracellularly-expressed antibodies of the various formats can be targeted into cellular compartments, e.g., the cytoplasm, the nucleus, the mitochondria, etc., by expressing them as fusions with the various known intracellular leader sequences. See Bradbury et al., Antibody Engineerinq, Borrebaeck, Editor, Vol. 2, pp. 295-361, IRL Press (1995). In particular, the ScFv format appears to be particularly suitable for cytoplasmic targeting.

Antibody types include, but are not limited to, polyclonal, monoclonal, chimeric, single-chain, Fab fragments and an Fab expression library. Various procedures known in the art may be used for the production of polyclonal antibodies to a target protein. For production of the antibody, various host animals can be immunized by injection with the target protein, such host animals include, but are not limited to, rabbits, mice, rats, etc. Various adjuvants can be used to increase the immunological response, depending on the host species and include, but are not limited to, Freund's (complete and incomplete); mineral gels, such as aluminum hydroxide; surface active substances, such as lysolecithin, pluronic polyols, polyanions, peptides, oil emulsions, dinitrophenol; and potentially useful human adjuvants, such as Bacillus Calmette-Guerin (BCG) and corynebacterium parvum.

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein, Nature, Vol. 256, pp. 495-497 (1975), the trioma technique, the human B-cell hybridoma technique [see Kozbor et al., Immunol. Today, Vol. 4, p. 72 (1983)] and the EBV hybridoma technique to produce human monoclonal antibodies [see Cole et al., Monoclonal Antibodies and Cancer Therapy, Alan R. Liss, Inc., pp. 77-96 (1985)]. In an additional embodiment of the invention, monoclonal antibodies can be produced in germ-free animals utilizing recent technology. See PCT/US90/02545.

According to the invention, human antibodies may be used and can be obtained by using human hybridomas [see Cote et al., Proc. Natl. Acad. Sci. USA, Vol. 80, pp. 2026-2030 (1983)], or by transforming human B cells with EBV virus in vitro [see Cole et al. (1985), supra]. In fact, according to the invention, techniques developed for the production of “chimeric antibodies” [see Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452454 (1985)] by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display. See Marks et al., J. Biol. Chem., Vol. 267, pp. 16007-16010 (1992). Using this technique, libraries of up to 10¹² different antibodies have been expressed on the surface of fd filamentous phage, creating a “single pot” in vitro immune system of antibodies available for the selection of monoclonal antibodies. See Griffiths et al., EMBO J., Vol. 13, pp. 3245-3260 (1994). Selection of antibodies from such libraries can be done by techniques known in the art, including contacting the phage to immobilized target protein, selecting and cloning phage bound to the target and subcloning the sequences encoding the antibody variable regions into an appropriate vector expressing a desired antibody format.

According to the invention, techniques described for the production of single-chain antibodies (see U.S. Pat. No. 4,946,778) can be adapted to produce single-chain antibodies specific to the target protein. An additional embodiment of the invention utilizes the techniques described for the construction of Fab expression libraries [see Huse et al., Science, Vol. 246, pp. 1275-1281 (1989)] to allow rapid and easy identification of monoclonal Fab fragments with the desired specificity for the target protein.

Antibody fragments that contain the idiotypes of the target protein can be generated by techniques known in the art. For example, such fragments include, but are not limited to, the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent and Fv fragments.

In the production of antibodies, screening for the desired antibody can be accomplished by techniques known in the art, e.g., ELISA. To select antibodies specific to a target protein, one may assay generated hybridomas or a phage display antibody library for an antibody that binds to the target protein.

Methods of Modifying Protein Activities

Methods of directly modifying protein activities include, inter alia, dominant negative mutations, specific drugs or chemical moieties and also the use of antibodies, as previously discussed.

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target. See Hershkowitz, Nature, Vol. 329, pp. 219-222 (1987). In the case of active monomeric forms, over expression of an inactive form can cause competition for natural substrates or ligands sufficient to significantly reduce net activity of the target protein. Such over expression can be achieved by, e.g., associating a promoter, preferably a controllable or inducible promoter, or also a constitutively expressed promoter, of increased activity with the mutant gene. Alternatively, changes to active site residues can be made so that a virtually Irreversible association occurs with the target ligand. Such can be achieved with certain tyrosine kinases by careful replacement of active site serine residues. See Perimutter et al., Curr. Opin. Immunol., Vol. 8, pp. 285-290 (1996).

In the case of active multimeric forms, several strategies can guide selection of a dominant negative mutant. Multimeric activity can be decreased in a controlled or saturating manner by expression of genes coding exogenous protein fragments that bind to multimeric association domains and prevent multimer formation. Alternatively, controllable or saturating over-expression of an inactive protein unit of a particular type can tie up wild-type active units in inactive multimers, and thereby decrease multimeric activity. See Nocka et al., EMBO J., Vol. 9, pp. 1805-1813 (1990). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes.

For a further example, proteins involved in cellular mechanisms, such as cellular motility, the mitotic process, cellular architecture and so forth, are typically composed of associations of many subunits of a few types. These structures are often highly sensitive to disruption by inclusion of a few monomeric units with structural defects. Such mutant monomers disrupt the relevant protein activities and can be expressed in a cell in a controlled or saturating manner.

In addition to dominant negative mutations, mutant target proteins that are sensitive to temperature (or other exogenous factors) can be found by mutagenesis and screening procedures that are well-known in the art.

Also, one of skill in the art will appreciate that expression of antibodies binding and inhibiting a target protein can be employed as another dominant negative strategy.

Modifying Proteins with Small Molecule Drugs

Finally, activities of certain target proteins can be modified or perturbed in a controlled or a saturating manner by exposure to exogenous drugs or ligands. Since the methods of this invention are often applied to testing or confirming the usefulness of various drugs to treat cancer, drug exposure is an important method of modifying/perturbing cellular constituents, both mRNAs and expressed proteins. In a preferred embodiment, input cellular constituents are perturbed either by drug exposure or genetic manipulation, such as gene deletion or knockout; and system responses are measured by gene expression technologies, such as hybridization to gene transcript arrays (described in the following).

In a preferable case, a drug is known that interacts with only one target protein in the cell and alters the activity of only that one target protein, either increasing or decreasing the activity. Graded exposure of a cell to varying amounts of that drug thereby causes graded perturbations of network models having that target protein as an input. Saturating exposure causes saturating modification/perturbation. For example, Cyclosporin A is a very specific regulator of the calcineurin protein, acting via a complex with cyclophilin. A titration series of Cyclosporin A therefore can be used to generate any desired amount of inhibition of the calcineurin protein. Alternately, saturating exposure to Cyclosporin A will maximally inhibit the calcineurin protein.

The present invention further relates to a diagnostic kit for predicting cancer patients at increased risk for bone metastasis by detecting overexpression of a calcium-binding protein in a cancerous tissue sample or blood sample from the patient.

In a preferred embodiment, a test kit for use in determining which patient with non-skeletal cancer, will be likely to develop bone metastasis; is provided which comprises the reagent able to determine the level or the presence of one or more calcium binding proteins as described above in a container suitable for contacting the tissue sample or body fluid, together with instructions for interpreting the results. Most preferably the reagent comprises an antibody able to specifically bind to the one or more calcium binding protein.

The present invention further relates to a diagnostic kit for predicting cancer patients at increased risk for bone metastasis by detecting the presence of MRP-14 in a cancerous tissue sample or plasma from the patient, especially wherein the kit comprises an antibody for MRP-14.

Computer Implementations

In some embodiments, the computation steps of the previous methods are implemented on a computer system or on one or more networked computer systems in order to provide a powerful and convenient facility for forming and testing models of biological systems. The computer system may be a single hardware platform comprising internal components and being linked to external components. The internal components of this computer system include processor element interconnected with a main memory. For example computer system can be an Intel Pentium based processor of 200 Mhz or greater clock rate and with 32 MB or more of main memory.

The external components include mass data storage. This mass storage can be one or more hard disks, which are typically packaged together with the processor and memory. Typically, such hard disks provide for at least 1 GB of storage. Other external components include user interface device, which can be a monitor and keyboards, together with pointing device, which can be a “mouse” or other graphic input devices. Typically, the computer system is also linked to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on mass storage. Alternatively, the software components may be stored on removable media such as floppy disks or CD-ROM (not illustrated). The software component represents the operating system, which is responsible for managing the computer system and its network interconnections. This operating system can be, e.g., of the Microsoft Windows family, such as Windows 95, Windows 98 or Windows NT; or a Unix operating system, such as Sun Solaris. Software include common languages and functions conveniently present on this system to assist programs implementing the methods specific to this invention. Languages that can be used to program the analytic methods of this invention include C, C++ or, less preferably, JAVA.

Preferably, the methods of this invention are programmed in mathematical software packages which allow symbolic entry of equations and high-level specification of processing, including algorithms to be used, thereby freeing a user of the need to procedurally program individual equations or algorithms. Such packages include, e.g., Matlab from Mathworks (Natick, Mass.), Mathematica from Wolfram Research (Champaign, Ill.) and MathCAD from Mathsoft (Cambridge, Mass.).

In some embodiments, the analytic software component actually comprises separate software components which interact with each other. Analytic software represents a database containing all data necessary for the operation of the system. Such data will generally include, but is not necessarily limited to, results of prior experiments, genome data, experimental procedures and cost and other information which will be apparent to those skilled in the art. Analytic software includes a data reduction and computation component comprising one or more programs which execute the analytic methods of the invention.

Analytic software also includes a user interface which provides a user of the computer system with control and input of test network models, and, optionally, experimental data. The user interface may comprise a drag-and-drop interface for specifying hypotheses to the system. The user interface may also comprise means for loading experimental data from the mass storage component, e.g., the hard drive; from removable media, e.g., floppy disks or CD-ROM; or from a different computer system communicating with the instant system over a network, e.g., a local area network, or a wide area communication network, such as the Internet.

Alternative systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### REFERENCES CITED

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes.

The present invention is not to be limited in terms of the particular embodiments described in this application, which are intended as single illustrations of individual aspects of the invention.

Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

### EXAMPLE 1

A study was performed to identify proteins that predict bone metastasis in patients presenting with lung and prostate cancer. In this study, 60 patients were selected, 30 with bone mets and 30 without bone mets, for each type of cancer. Plasma was collected at three time points for each cancer; 1) at cancer diagnosis, 2) at the time of diagnosis of bone metastases and 3) in late stage IV disease and tumor tissue was taken. The presence of MRP-14 was detected by immunohistological (IHC) methods. Of the tumors which produced bone mets, 68% of the tumors were positive for MRP-14 by IHC. Of the tumors which did not produce bone mets 0% were positive for MRP-14 by IHC and in a control sample of normal tissue 0% were positive for MRP-14 by IHC. The conclusion is that, in tissue samples taken from patients with lung and prostate cancer, at the time of diagnosis, it was possible to determine 68% of those which would metastsize to bone by staining for MRP-14 by the use of immunohistological staining methods. The tissue samples which stained positive were the only ones to form bone mets and none of the tissue samples from those tumors which did not form bone mets, or from the normal control tissues, were found to be positive by this method.

### EXAMPLE 2

In another study the levels of MRP-14 were quantitatively determined by the use of standard ELISA quantitative assays methods in the plasma of patients with breast cancer. Patients with and without bone mets were included. In this study it was determined that the average level of MRP-14 in sera of 29 patients with breast cancer with bone metastasis was 10.21 with a standard deviation of 1.97 and the average level of MRP-14 in 29 patients with breast cancer who did not have bone metastasis was 6.51 with a standard deviation of 0.86. The specific results are shown in Table 1 below.

TABLE 1

| | | average level | standard | |
|---|---|---|---|---|
| Sample number | Patients with bone mets | (ng/ml) | deviation | CV |
| 1 | sera breast cancer/bone mets | 2.131 | 0.478 | 22.44 |
| 2 | sera breast cancer/bone mets | 10.21 | 4.522 | 44.28 |
| 3 | sera breast cancer/bone mets | 5.517 | 0.559 | 10.14 |
| 4 | sera breast cancer/bone mets | 7.773 | 3.269 | 42.05 |
| 5 | sera breast cancer/bone mets | 35.67 | 7.176 | 20.17 |
| 6 | sera breast cancer/bone mets | 2.835 | 0 | 0 |
| 7 | sera breast cancer/bone mets | 3.158 | 0.238 | 7.540 |
| 8 | sera breast cancer/bone mets | 1.475 | 0.130 | 8.803 |
| 9 | sera breast cancer/bone mets | 22.87 | 0.076 | 0.333 |
| 10 | sera breast cancer/bone mets | 2.582 | 0.179 | 6.933 |
| 11 | sera breast cancer/bone mets | 6.070 | 1.985 | 32.70 |
| 12 | sera breast cancer/bone mets | 5.066 | 0.541 | 10.69 |
| 13 | sera breast cancer/bone mets | 39.42 | 6.454 | 16.37 |
| 14 | sera breast cancer/bone mets | 2.912 | 0.705 | 24.22 |
| 15 | sera breast cancer/bone mets | 2.230 | 0.219 | 9.825 |
| 16 | sera breast cancer/bone mets | 1.941 | 0.608 | 31.34 |
| 17 | sera breast cancer/bone mets | 7.838 | 2.722 | 34.73 |
| 18 | sera breast cancer/bone mets | 4.729 | 1.429 | 30.22 |
| 19 | sera breast cancer/bone mets | 3.626 | 1.178 | 32.50 |
| 20 | sera breast cancer/bone mets | 14.95 | 4.028 | 26.94 |
| 21 | sera breast cancer/bone mets | 13.90 | 2.109 | 15.17 |
| 22 | sera breast cancer/bone mets | 5.620 | 3.958 | 70.43 |
| 23 | sera breast cancer/bone mets | 38.99 | 3.955 | 10.14 |
| 24 | sera breast cancer/bone mets | 4.047 | 0.662 | 16.37 |
| 25 | sera breast cancer/bone mets | 1.376 | 0.430 | 31.23 |
| 26 | sera breast cancer/bone mets | 3.290 | 1.002 | 30.44 |
| 27 | sera breast cancer/bone mets | 8.416 | 1.845 | 21.93 |
| 28 | sera breast cancer/bone mets | 34.96 | 3.383 | 9.675 |
| 29 | sera breast cancer/bonemets | 2.547 | 0.249 | 9.764 |
| | TOTAL | 10.21 | 1.97 | |
| | | average level | standard | |
| Patient number | Patients without bone mets | (ng./ml. | deviation | CV |
| 1 | sera breast cancer/without bone mets | 3.163 | 1.439 | 45.48 |
| 2 | sera breast cancer/without bone mets | 1.603 | 0.110 | 6.854 |
| 3 | sera breast cancer/without bone mets | 15.95 | 1.352 | 8.480 |
| 4 | sera breast cancer/without bone mets | 1.546 | 0.170 | 10.98 |
| 5 | sera breast cancer/without bone mets | 9.141 | 3.142 | 34.37 |
| 6 | sera breast cancer/without bone mets | 5.207 | 2.165 | 41.57 |
| 7 | sera breast cancer/without bone mets | 5.842 | 1.462 | 25.03 |
| 8 | sera breast cancer/without bone mets | 7.418 | 0.078 | 1.054 |
| 9 | sera breast cancer/without bone mets | 6.296 | 1.821 | 28.92 |
| 10 | sera breast cancer/without bone mets | 3.249 | 0.526 | 16.18 |
| 11 | sera breast cancer/without bone mets | 5.483 | 0.324 | 5.915 |
| 12 | sera breast cancer/without bone mets | 4.877 | 1.103 | 22.63 |
| 13 | sera breast cancer/without bone mets | 10.66 | 1.463 | 13.73 |
| 14 | sera breast cancer/without bone mets | 3.863 | 1.474 | 38.15 |
| 15 | sera breast cancer/without bone mets | 11.08 | 1.330 | 12.00 |
| 16 | sera breast cancer/without bone mets | 5.490 | 0.432 | 7.876 |
| 17 | sera breast cancer/without bone mets | 1.426 | 0.400 | 28.03 |
| 18 | sera breast cancer/without bone mets | 23.51 | 0.696 | 2.960 |
| 19 | sera breast cancer/without bone mets | 2.835 | 0.099 | 3.504 |
| 20 | sera breast cancer/without bone mets | 3.897 | 0.861 | 22.19 |
| 21 | sera breast cancer/without bone mets | 5.330 | 0.423 | 7.933 |
| 22 | sera breast cancer/without bone mets | 2.110 | 0.488 | 23.14 |
| 23 | sera breast cancer/without bone mets | 23.71 | 2.479 | 10.45 |
| 24 | sera breast cancer/without bone mets | 2.372 | 0.608 | 25.62 |
| 25 | sera breast cancer/without bone mets | 1.016 | 0.065 | 6.428 |
| 26 | sera breast cancer/without bone mets | 7.958 | 0.130 | 1.632 |
| 27 | sera breast cancer/without bone mets | 2.235 | 0.599 | 26.81 |
| 28 | sera breast cancer/without bone mets | 6.568 | 2.654 | 40.42 |
| 29 | sera breast cancer/without bone mets | 5.015 | 0.081 | 1.611 |
| | TOTAL | 6.51 | 0.86 | |

The conclusion of this study is that, in patients with breast cancer the levels of MPR-14 significantly higher in those patients who developed bone metastasis as compared with those patients whose breast cancer does not metastasize to bone. Thus, the level of this MRP-14 in a breast cancer patient's blood can be used as an indicator of which patients have cancers that will metastasize to bone.

1. A method of inhibiting bone metastasis in a patient diagnosed with a non-skeletal cancer, which comprises
(a) testing a sample of tissue or body fluid from the patient for the presence of one or more calcium binding proteins selected from the group consisting of migration inhibitory factor related protein 14 (MRP-14), S100A1 to 8, S10010-113, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, and Calcium- and Integrin-Binding Protein, and, if one or more calcium binding proteins are detected,
(b) treating the patient with a bone metastasis inhibiting therapy.

2. A method of inhibiting bone metastasis in a patient diagnosed with a non-skeletal cancer, which comprises
(a) testing a cancerous tissue sample or blood from the patient for the presence of a calcium binding protein, and, if a calcium binding protein is detected,
(b) treating the patient with a bone metastasis inhibiting therapy.

3. A method of claim 1 or 2, wherein the calcium binding protein is a member of the S100 protein family.

4. A method of claim 1 or 2, wherein the calcium binding protein is selected from the group consisting of MRP-14, S100A1 to 8, S10010-113, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, and Calcium- and Integrin-Binding Protein.

5. A method of claim 1 wherein the non-skeletal cancer is breast cancer, genitourinary cancer, lung cancer, gastrointestinal cancer, epidermoid cancer, melanoma, ovarian cancer, prostate cancer, pancreas cancer, neuroblastoma, head and/or neck cancer, bladder cancer, renal cancer, brain cancer or gastric cancer.

6. A method of claim 5, wherein the non-skeletal cancer is lung cancer.

7. A method of claim 5, wherein the non-skeletal cancer is breast cancer.

8. A method of claim 1 wherein the bone metastasis inhibiting therapy includes treatment with a bisphosphonate in its acid or salt form.

9. A method of claim 1, wherein the bisphosphonate is zolendronic acid or a salt thereof.

10. A method of inhibiting bone metastasis in a patient diagnosed with a non-skeletal cancer, which comprises
(a) testing a sample of tissue or body fluid from the patient for the presence of the calcium binding protein MAP-14, and, if MRP-14 is detected,
(b) treating the patient with a bone metastasis inhibiting therapy.

11. A method of inhibiting bone metastasis in a patient diagnosed with a non-skeletal cancer, which comprises
(a) testing a cancerous tissue sample or blood from the patient for the presence of MAP-14, and, if migration inhibitory factor related protein 14 is detected,
(b) treating the patient with a bone metastasis inhibiting therapy.

12. A method of claim 10 or 11, wherein the bone metastasis inhibiting therapy includes treatment with a bisphosphonate in its acid or salt form.

13. A method of claim 12, wherein the bisphosphonate is zolendronic acid, or a pharmaceutically acceptable salt thereof.

14. A method of claim 10 or 11, wherein the non-skeletal cancer is breast cancer, genitourinary cancer, lung cancer, gastrointestinal cancer, epidermoid cancer, melanoma, ovarian cancer, prostate cancer, pancreas cancer, neuroblastoma, head and/or neck cancer, bladder cancer, renal, brain or gastric cancer.

15. A method of claim 14, wherein the non-skeletal cancer is lung cancer.

16. A method of claim 14, wherein the non-skeletal cancer is breast cancer.

17. A method of claim 1, wherein the presence of a calcium binding protein is determined by mass spectroscopy.

18. A method of claim 1 wherein the presence of a calcium binding protein is determined by Western blot or ELISA.

19. A method of claim 18, wherein a labeled probe specific for the calcium binding protein is used.

20. A method of claim 19, wherein the labeled probe is an antibody or a radiolabeled binding partner.

21. A method of claim 20, wherein the antibody is a monoclonal antibody.

22. A method of claim 21, wherein the antibody is the anti-MAP14 antibody MAC387.

23. A method of claim 1, wherein the presence of one or more calcium binding proteins is determined by measuring the levels of expression of one or more genes encoding said one or more calcium binding proteins.

24. A method of claim 23, wherein the level of expression is detected by techniques selected from the group consisting of microarray analysis, Northern blot analysis, reverse transcription PCR and real time quantitative PCR.

25. A method for determining, which patient, diagnosed with a non-skeletal cancer, will be likely to develop bone metastasis; comprising:
a) obtaining a sample of tissue or body fluid from the said patient;
b) determining whether the levels of one or more calcium binding proteins selected from the group consisting of migration inhibitory factor related protein 14 (MAP-14), S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, and Calcium- and Integrin-Binding Protein are increased in the sample of tissue or body fluid;
c) determining that the said patient is in a high risk group for developing bone metastasis if the levels of the one or more calcium binding proteins are increased; and
d) determining that the said patient is in a low risk group for developing bone metastasis if the levels of the one or more calcium binding proteins are not increased.

26. A method for determining, which patient, diagnosed with a non-skeletal cancer, will be likely to develop bone metastasis; comprising:
a) obtaining a sample of tissue or body fluid from the said patient;
b) determining the presence of one or more calcium binding proteins in the said sample of tissue or body fluid;
c) determining that the said patient is in a high risk group for developing bone metastasis if the presence of the one or more calcium binding proteins is detected; and
d) determining that the said patient is in a low risk group for developing bone metastasis if the one or more calcium binding proteins are not detected.

27. A method for determining, which patient, diagnosed with a non-skeletal cancer, will be likely to develop bone metastasis; comprising:
a) determining in vitro or ex vivo whether the levels of one or more calcium binding proteins are increased in a sample of tissue or body fluid of said patient;
b) determining that the said patient is in a high risk group for developing bone metastasis if the levels of the one or more calcium binding proteins are increased; and
c) determining that the said patient is in a low risk group for developing bone metastasis if the levels of the one or more calcium binding proteins are not increased.

28. A method for determining, which patient, diagnosed with a non-skeletal cancer, will be likely to develop bone metastasis; comprising:
a) determining in vitro or ex vivo the presence of one or more calcium binding proteins in a sample of tissue or body fluid of said patient;
b) determining that the said patient is in a high risk group for developing bone metastasis if the one or more calcium binding proteins are detected; and
c) determining that the said patient is in a low risk group for developing bone metastasis if the one or more calcium binding protein are not detected.

29. A method of claim 25, 26, 27 or 28, wherein the calcium binding protein is a member of the S100 protein family.

30. A method of claim 25, 26, 27 or 28, wherein the calcium binding protein is selected from the group consisting of MAP-14, S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, and Calcium- and Integrin-Binding Protein.

31. A method of claim 25 wherein the said sample of tissue or body fluid is selected from the group consisting of a tissue biopsy, blood, serum, plasma, lymph, ascitic fluid, cystic fluid, urine, cerebro-spinal fluid (CSF), salvia or sweat.

32. A method of claim 25 wherein the non-skeletal cancer is breast cancer, genitourinary cancer, lung cancer, gastrointestinal cancer, epidermoid cancer, melanoma, ovarian cancer, prostate cancer, pancreas cancer, neuroblastoma, head and/or neck cancer, bladder cancer, renal, brain or gastric cancer.

33. A method of claim 32, wherein the non-skeletal cancer is lung cancer.

34. A method of claim 32, wherein the non-skeletal cancer is breast cancer.

35. A method of claim 25 wherein the step of determining the levels of or the presence of one or more calcium binding proteins in the said sample of tissue or body fluid is performed by measuring the levels of the said one or more calcium binding proteins by means of mass spectrometry.

36. A method of claim 25 wherein the levels of or the presence of the one or more calcium binding proteins in the said sample of tissue or body fluid is performed by measuring the levels of said one or more calcium binding proteins by means of a reagent which specifically binds to the protein.

37. A method of claim 36, wherein the reagent is a labeled probe specific for the said calcium binding protein.

38. A method of claim 37, wherein the reagent comprises an antibody.

39. A method of claim 38, wherein the reagent is a monoclonal antibody.

40. A method of claim 25, wherein the levels or the presence of one or more calcium-binding proteins are determined by measuring the levels of expression of one or more genes encoding said one or more calcium-binding proteins.

41. The method of claim 40, wherein the level of expression is detected by techniques selected from the group consisting of microarray analysis, Northern blot analysis, reverse transcription PCR and real time quantitative PCR.

42. A method for screening for an agent useful in treating bone metastasis in a patient diagnosed with a non-skeletal cancer, comprising:
(a) administering a candidate agent to a non-human test animal which is predisposed to be affected or being affected by bone metastasis;
(b) administering the candidate agent of (a) to a matched control non-human animal not predisposed to be affected or being affected by bone metastasis;
(c) determining the levels of one or more calcium-binding proteins selected from migration inhibitory factor related protein 14 (MRP-14), S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, Calcium- and Integrin-Binding Protein in a sample obtained from the animal of (a) and (b);
(d) comparing the levels determined in (c), wherein a decrease in the levels of the said one or more calcium-binding proteins indicates that the candidate agent is an agent useful in treating bone metastasis.

43. A method of claim 42, wherein the one or more calcium-binding protein is selected from MRP-14, S100A1 to 8, S100A10-13, S100P, Calbindin 1 to 3, Calcium-Binding Protein 1 to 5, Histidine-Rich Calcium-Binding Protein, Annexin A6, Secreted Modular Calcium-Binding Protein 2, Reticulocalbin 1, Caltractin, Grancalcin, Calcium- and Integrin-Binding Protein.

44. A method of claim 43, wherein the calcium-binding protein is MAP-14.

45. A method of claim 42 wherein the sample is a sample of tissue or body fluid.

46. A method of claim 42 wherein the levels of the one or more calcium binding proteins are determined by mass spectroscopy.

47. A method of claim 42 wherein the levels of the one or more calcium-binding proteins are determined by Western blot or ELISA

48. A method of claim 47, wherein a labeled probe specific for the one or more calcium binding proteins is used.

49. A method of claim 48, wherein the labeled probe is an antibody or a radiolabeled binding partner.

50. A method of claim 49 wherein the antibody is a monoclonal antibody.

51. A method of claim 42 wherein the levels of the one or more calcium binding proteins are determined by measuring the level of expression of one or more genes encoding said one or more calcium-binding proteins.

52. A method of claim 51, wherein the levels of expression are detected by techniques selected from the group consisting of Microarray analysis, Northern blot analysis, reverse transcription PCR and real time quantitative PCR.

53. A method of claim 42, wherein the agent is selected from the group consisting of antisense nucleotides, ribozymes and double stranded RNAs.

54. A test kit for use in determining which patient with non-skeletal cancer, will be likely to develop bone metastasis; comprising the reagent of claim 36 in a container suitable for contacting the said body fluid, with instructions for interpreting the results.

55. A test kit of claim 54, wherein the reagent comprises an antibody, and wherein said antibody specifically binds with the said calcium binding protein.

56. A test kit of claim 54 comprising an antibody able to contact the level and/or presence of MRP-14 in a sample of tissue or body fluid.

57. The method of claim 1, wherein the tissue sample is a cancerous tissue sample.

58. The method of claim 1, wherein the body fluid is blood.
